(19) United States
(12) Patent Application Publication
(10) Pub. No.: US 2015/0203837 A1
(43) Pub. Date: Jul. 23, 2015

(54) FUSION PROTEIN FOR ACTIVATING RTK SIGNAL TRANSMISSION BY MEANS OF LIGHT, AND USE THEREOF

(71) Applicant: KOREA ADVANCED INSTITUTE OF SCIENCE AND TECHNOLOGY, Daejeon (KR)

(72) Inventors: Won Do Heo, Daejeon (KR); Ki Young Chang, Daejeon (KR); Do Yeon Woo, Daejeon (KR)

(21) Appl. No.: 14/383,721

(22) PCT Filed: Mar. 7, 2013
(86) PCT No.: PCT/KR2013/001847

§ 371 Date: Jan. 7, 2015

(30) Foreign Application Priority Data
Mar. 7, 2012 (KR) 10-2012-0023638

(51) Int. Cl. C12N 13/00; C12Q 1/48; C12N 9/12

(52) U.S. Cl. CPC C12N 13/00; C12N 9/12; C12Q 1/485; C12Y 207/10001

(57) ABSTRACT

The present invention relates to a fusion protein for reversibly activating RTK signal transmission by means of light and relates to a use thereof, and the invention provides a fusion protein in which a light induced dimer forming protein is coupled to the C-terminal of a receptor tyrosine kinase (RTK) protein or a modified RTK protein which has been modified so as to eliminate a ligand binding site of the RTK protein or ensure that the ligand does not bind.

### FIELD OF THE INVENTION

The present invention relates to a fusion protein and use thereof, in more detail to a fusion protein activating RTK signaling by light irradiation and use thereof.

### BACKGROUND OF THE INVENTION

Receptor tyrosine kinases (RTKs) are cell membrane receptors which bind to various peptide such as growth factors, cytokines and hormones. If the above-mentioned peptides bind to the extracellular domain of RKT, dimerization of RTK occurs and then the autophosphorylation of tyrosine existing in the intracellular domain occurs (activation of the receptor). As a result, a series of signal cascades result in metastasis, angiogenesis, the inhibition of apoptosis and the progression of various cancer as well as DNA synthesis, cell growth, and proliferation of normal cells. Thus, a variety of studies regarding the development of various therapeutics for treating cancer targeting the RTK signaling have been conducted. For examples, Korean Patent Gazette No. 10-2006-0132965 and No. 10-1996-7002442 disclose methods for treating solid tumors and angiogenesis-related diseases through the inhibition of tyrosine kinase activity by particular compounds. In addition, the treatment of NSCLC (non-small cell lung cancer) by the inhibition of RTK signaling (Niels et al., International Journal of Cancer, 119(4), 727-734, 2006) and the treatment of ovarian cancer by the inhibition MAPK and PI3K signaling which are downstream signaling pathways of the RTK (Helen et al., Biochemical Pharmacology, 72(8), 941-948, 2006; Jeffrey et al., Nature Reviews Cancer, 9, 550-562, 2009) were reported.

The dimerization by chemical inducers of dimerization (CID) means a method for investigating activity of proteins using organic compounds having low molecular weight. Properties of natural compounds such as FK506, cyclosporine and rapamycin and synthetic derivatives which bind to immunophillin domain of FKBP12 (FK506-binding protein 12), cyclophilin and FKBP12-rapamycin-binding (FRB) domain, respectively were used for studying signaling mechanism mediated by the protein by inducing the dimerization of target proteins by treating the compounds such as rapamycin after fusing the target protein to the above-mentioned proteins such as the immunophilin domain (Crabtree et al., Trends Biochem. Sci., 21:418-422, 1996). The properties of the CIDs were used for investigating signal transduction pathways mediated by T-cell receptor (Spencer et al., Science, 262:1019-1024, 1993; Pruschy et al., Chem. Biol., 1:163-172, 1994), Fas (Belshaw et al., Chem. Biol., 3:731-738, 1996; Spence et al., Curr. Biol., 6:839-847, 1996), cadherin (Yap et al., Curr. Biol., 7:308-315, 1997), and erythropoietin receptor (Blau et al., Proc. Natl. Acad. Sci. USA, 94:3076-3081, 1997). In addition, signal transduction pathways of Src (Spencer et al., Proc. Natl. Acad. Sci. USA, 92:9805-9809, 1995), Sos (Holsinger et al., Proc. Natl. Acad. Sci. USA, 92: 9810-9814, 1995), Raf (Luo et al., Nature, 383:181-185, 1996), and Zap (Graef et al., EMBO J., 16:5618-5628, 1997) which are intracellular proteins were analyzed, too. However, compounds known as CIDs and proteins which bind to the CIDs are very limited and it is difficult to regulate the dimerization by the CIDs and the level of kinase activity, and the CIDs may be involved in various signaling processes of a cell or an organism as well as the dimerization of proteins thus the usage of CIDs is limited.

### SUMMARY OF THE INVENTION

However, the afore-mentioned methods have some disadvantages that they have low industrial applicability in terms of cost and time, they can only be performed in vitro as well as they cause irreversible reactions because they only use in vitro level analysis for investigating RTK signaling pathway and in vitro level screening of inhibitors capable of inhibiting specifically the RTK signaling pathway.

The present invention to solve various problem including the afore-mentioned problems is intended to provide a fusion protein capable of being used in vitro as well as in vivo condition and activating RTK signaling pathway selectively and reversibly by light irradiation and uses thereof. In addition, the present invention is intended to provide a method for screening a substance capable of regulating RTK signaling and being a therapeutic candidate for treating various diseases including cancer thereby. However, these objects are illustrative, and thus the scope of the present invention is not limited thereto.

In an aspect of the present invention, a fusion protein in which a light-induced dimerized protein is fused to C-terminus of receptor tyrosine kinase (RTK) or RTK mutant protein whose ligand binding domain is deleted or mutated so as not to bind to a ligand is provided.

According to the fusion protein, the receptor tyrosine kinase may be epidermal growth factor receptor (EGFR, RTK class I), insulin receptor (IR, RTK class II), platelet-derived growth factor receptor (PDGF, RTK class III), fibroblast growth factor receptor (FGFR, RTK class IV), vascular endothelial cell growth factor receptor (VEGFR, RTK class V), hepatocyte growth factor receptor (HGFR, RTK class VI), tropomyosin-receptor-kinase receptor (TRKR, RTK class VII), EPH receptor (RTK class VIII), AXL receptor (RTK class IX), LKT receptor (RTK class X), TIER receptor (RTK class XI), receptor tyrosine kinase-like orphan receptor (RTK class XII), discoidin domain receptor (RTK class XIII), RET receptor (RTK class XIV), KLG receptor (RTK class XV), related to receptor tyrosine kinase (RYK) receptor (RTK class XVI), or Muscle-Specific Kinase (MuSK) Receptor (RTK class XVII).

According to the fusion protein, the light-induced dimerized protein may be a light-induced heterodimerized protein and/or a light-induced homodimerized protein. The light-induced heterodimerized protein may be CIB (cryptochrome-interacting basic-helix-loop-helix protein), CIBN (N-terminal domain of CIB), Phy (phytochrome), PIF (phytochrome interacting factor), FKF1 (Flavin-binding, Kelch repeat, F-box 1), GIGANTEA, CRY (chryptochrome) or PHR (phytolyase homolgous region). The light-induced homodimerized protein maybe CRY or PHR. Although the CRY and the PHR are known to form homodimers regardless of light irradiation, it was confirmed that both proteins form homodimers when light irradiated by the present inventors. Therefore, the CRY or the PHR are proteins capable of forming homodimers as well as heterodimers.

In addition, a fluorescent protein may be linked to the fusion protein. In this case, the fluorescent protein may be green fluorescent protein (GFP), yellow fluorescent protein (YFP), red fluorescent protein (RFP), orange fluorescent protein, cyan fluorescent protein (CFP), blue fluorescent protein (BFP), or tetracysteine fluorescent motif. The green fluorescent protein may be EGFP (enhanced green fluorescent protein), Emerald (Tsien, Annu. Rev. Biochem., 67: 509-544, 1998), Superfolder (Pedelacq et al., Nat. Biotech., 24: 79-88 , 2006), GFP (Prendergast et al., Biochem., 17(17): 3448-3453, 1978), Azami Green (Karasawa, et al., J. Biol. Chem., 278:34167-34171, 2003) , TagGFP (Evrogen, Russia), TurboGFP (Shagin et al., Mol. Biol. Evol., 21(5): 841-850, 2004), ZsGreen (Matz et al., Nat. Biotechnol., 17: 969-973, 1999) or T-Sapphire (Zapata-Hommer et al, BMC Biotechnol., 3: 5, 2003). The yellow fluorescent protein may be EYFP (enhanced yellow fluorescent protein, Tsien, Annu. Rev. Biochem., 67: 509-544, 1998), Topaz (Hat et al., Ann. NY Acad. Sci., 1: 627-633, 2002), Venus (Nagai et al., Nat. Biotechnol., 20(1): 87-90, 2002), mCitrine (Griesbeck et al., J. Biol. Chem., 276: 29188-29194, 2001), Ypet (Nguyet and Daugherty, Nat. Biotechnol., 23(3): 355-360 , 2005), TagYFP (Evrogen, Russia), PhiYFP (Shagin et al., Mol. Biol. Evol., 21(5): 841-850, 2004), ZsYellow1 (Matz et al., Nat. Biotechnol., 17: 969-973, 1999), or mBanana (Shaner et al., Nat. Biotechnol., 22: 1567-1572, 2004). The red fluorescent protein may be mRuby (Kredel et al., PLoS ONE, 4(2): e4391, 2009), mApple (Shaner et al., Nat. Methods, 5(6): 545-551, 2008), mStrawberry (Shaner et al., Nat. Biotechnol., 22: 1567-1572, 2004) and AsRed2 (Shanner et al., Nat. Biotechnol., 22: 1567-1572, 2004) or mRFP (Campbell et al., Proc. Natl. Acad. Sci. USA, 99(12): 7877-7882, 2002). The orange fluorescent protein may be Kusabira Orange (Karawawa et al., Biochem. J. 381(Pt 1): 307-312, 2004), Kusabira Orange2 (MBL International Corp., Japan), mOrange (Shaner et al., Nat. Biotechnol., 22: 1567-1572, 2004), mOrange2 (Shaner et al., Nat. Biotechnol., 22: 1567-1572, 2004), dTomato (Shaner et al., Nat. Biotechnol., 22: 1567-1572, 2004), dTomato-Tandem (Shaner et al., Nat. Biotechnol., 22: 1567-1572, 2004), TagRFP (Merzlyak et al., Nat. Methods, 4(7): 555-557, 2007), TagRFP-T (Shaner et al., Nat. Methods, 5(6): 545-551, 2008), DsRed (Baird et al., Proc. Natl. Acad. Sci. USA, 97: 11984-11989, 1999), DsRed2 (Clontech, USA), DsRed-Express (Clontech, USA), DsRed-Monomer (Clontech, USA), or mTangerine (Shaner et al., Nat Biotechnol, 22: 1567-1572, 2004 above). The cyan fluorescent protein may be ECFP (enhanced cyan fluorescent protein, Cubitt et al., Trends Biochem. Sci., 20: 448-455, 1995), mECFP (Ai et al., Biochem. J., 400(3): 531-540, 2006), mCerulean (Koushik et al., Biophys. J., 91(12): L99-L101, 2006), CyPet (Nguyet and Daugherty, Nat. Biotechnol., 23 (3): 355-360, 2005), AmCyan1 (Matz et al., Nat. Biotechnol., 17: 969-973, 1999), Midori-Ishi Cyan (Karawawa et al., Biochem. J., 381(Pt 1): 307-312, 2004), TagCFP (Evrogen, Russia) or mTFP1, (Ai et al, Biochem. J., 400 (3): 531-540, 2006). The blue fluorescent protein may be EBFP (enhanced blue fluorescent protein, Clontech, USA), EBFP2 (Ai et al., Biochemistry, 46 (20): 5904-5910, 2007), Azurite (Mena et al., Nat. Biotechnol., 24: 1569-1571, 2006) or mTagBFP (Subach et al., Chem. Biol., 15(10): 1116-1124, 2008). The far red fluorescent protein may be mPlum (Wang et al., Proc. Natl. Acad. Sci. USA, 101: 16745-16749, 2004), mCherry (Shanner et al., Nat. Biotechnol., 22: 1567-1572, 2004), dKeima-Tandem (Kogure et al., Methods, 45(3): 223-226, 2008), JRed (Shagin et al., Mol. Biol. Evol., 21(5): 841-850, 2004), mRaspberry (Shanner et al., Nat. Biotechnol., 22: 1567-1572, 2004), HcRed1 (Fradkov et al., Biochem. J., 368(Pt 1): 17-21, 2002), HcRed-Tandem (Fradkov et al., Nat. Biotechnol., 22(3): 289-296, 2004), AQ143 (Shkrob et al., Biochem. J., 392: 649-654, 2005). The tetracysteine fluorescent motif may be a polypeptide including an amino acid sequence of Cys-Cys-Xaa-Xaa-Cys-Cys (SEQ ID NO: 1), wherein the Xaa is any one amino acid except cysteine.

In an aspect of the present invention, a polynucleotide encoding the fusion protein is provided.

In an aspect of the present invention, a vector comprising the polynucleotide is provided. The vector may be an expression vector capable of expressing the fusion protein.

In an aspect of the present invention, a transformed host cell transformed with the vector is provided. The transformed host cell may express the fusion protein within cytosol.

In an aspect of the present invention, a transgenic non-human animal expressing the fusion protein transformed with the vector is provided.

The transgenic non-human animal may be an insect, an annelid, a mollusk, a brachiopod, a nematode, a coelenterata, a sponge, a chordata or verterbrate, and the vertebrate may be a fish, an amphibian, a reptile, a bird or a mammal. The insect may be Drosophila, the nematode may be Caenorhabditis elegans (C. elegans), the fish may be a zebrafish, and the mammal may be a primate, a carnivore, an insectivore, a rodent, an artiodactyla, a perissodactyla, or an elephant, and the rodent may be a rat or a mouse.

In another aspect of the present invention, a transgenic plant expressing the fusion protein, which is transformed with the vector is provided. The transgenic plant may be a gymnosperm, or an angiosperm, and the angiosperm may be a monocotyledon or a dicotyledon. The monocotyledon may be a poaceae or a lilliaceae, or orchidaceae. The dicotyledon may be a leguminosae, a cucurbitaceae, a asteraceae, a solanaceae, a rosaceae, or a cruciferae. The leguminosae may be a soybean, a green bean, a pea, or a red bean. The cucurbitaceae may be a watermelon, a pumpkin, a cucumber, a melon or an oriental melon. The asteraceae may be a chrysanthemum, a lettuce, a dandelion, a crown daisy, or a mugwort. The solanaceae may be a tobacco, a pepper, an eggplant, or a tomato. The rosaceae may be an apple, a pear, a peach, a rose or a strawberry. The cruciferae may be a radish, a cabbage, a rapeseed, a leaf mustard, a horseradish or Arabidopsis thaliana (A. thaliana).

In another aspect of the present invention, a method for activating RTK in a cell reversibly, comprising preparing a transformed host cell by transforming a host cell with an expression vector comprising a gene construct including a promoter and a polynucleotide encoding a fusion protein in which a light-induced homodimerized protein is fused to C-terminus of receptor tyrosine kinase (RTK) or RTK mutant protein whose ligand binding domain is deleted or mutated so as not to bind to a ligand, wherein the polynucleotide is operably linked to the promoter; and irradiating light having wavelength capable of inducing the homodimerization of the lihgt-induced homodimerized protein is provided.

In another aspect of the present invention, a method for activating receptor tyrosine kinase (RTK) in an organ or a tissue of a plant or a non-human animal, comprising preparing a transgenic plant or a transgenic non-human animal by transforming a plant or the non-human animal with an expression vector comprising a gene construct including a promoter and a polynucleotide encoding a fusion protein in which a light-induced homodimerized protein is fused to C-terminus of receptor tyrosine kinase (RTK) or RTK mutant protein whose ligand binding domain is deleted or mutated so as not to bind to a ligand, respectively, wherein the polynucleotide is operably linked to the promoter; and irradiating light having wavelength capable of inducing the homodimerization of the light-induced homodimerized protein to the organ or the tissue is provided.

According to the method, the RTK may be epidermal growth factor receptor (EGFR, RTK class I), insulin receptor (IR, RTK class II), platelet-derived growth factor receptor (PDGF, RTK class III), fibroblast growth factor receptor (FGFR, RTK class IV), vascular endothelial cell growth factor receptor (VEGFR, RTK class V), hepatocyte growth factor receptor (HGFR, RTK class VI), tropomyosin-receptor-kinase receptor (TRKR, RTK class VII), EPH receptor (RTK class VIII), AXL receptor (RTK class IX), LKT receptor (RTK class X), TIER receptor (RTK class XI), receptor tyrosine kinase-like orphan receptor (RTK class XII), discoidin domain receptor (RTK class XIII), RET receptor (RTK class XIV), KLG receptor (RTK class XV), related to receptor tyrosine kinase (RYK) receptor (RTK class XVI), or Muscle-Specific Kinase (MuSK) Receptor, RTK class XVII).

According to the method, the light-induced homodimerized protein may be cryptochrome (CYR) or PHR (phytolyase homologous region).

According to the method, the non-human animal may be an insect, an annelid, a mollusk, a brachiopod, a nematode, a coelenterata, a sponge, a chordata or verterbrate, and the vertebrate may be a fish, an amphibian, a reptile, a bird or a mammal. The insect may be Drosophila, the nematode may be Caenorhabditis elegans (C. elegans), the fish may be a zebrafish, and the mammal may be a primate, a carnivore, an insectivore, a rodent, an artiodactyla, a perissodactyla, or an elephant, and the rodent may be a rat or a mouse.

According to the method, the fusion protein may further comprise a fluorescent protein. In this case, the fluorescent protein may fused to an N-terminus or to a C-terminus of the RTK or the light-induced dimerized protein fused to the RTK. The fluorescent protein is as described above.

In another aspect of the present invention, a method for screening an inhibitor candidate of RTK signaling comprising preparing a transformed host cell by transforming a host cell with an expression vector comprising a gene construct including a promoter and a polynucleotide encoding a fusion protein in which a light-induced homodimerized protein is fused to C-terminal of receptor tyrosine kinase (RTK) or RTK mutant protein whose ligand binding domain is deleted or mutated so as not to bind to a ligand, wherein the polynucleotide is operably linked to the promoter; treating candidate substances to culture media containing the transformed cell; irradiating light having wavelength capable of inducing the homodimerization of the lihgt-induced homodimerized protein to the transformed cell; and selecting a candidate substance inhibiting RTK signaling by the light irradiation significantly compared to a negative control.

According to the method, the candidate substance may be a peptide, a protein, a non-peptide compound, a synthetic compound, a fermentation product, cell extract, plant extract, animal tissue extracts or plasma.

According to the method, the non-human animal may be an insect, an annelid, a mollusk, a brachiopod, a nematode, a coelenterata, a sponge, a chordata or verterbrate, and the vertebrate may be a fish, an amphibian, a reptile, a bird or a mammal. The insect may be Drosophila, the nematode may be Caenorhabditis elegans (C. elegans), the fish may be a zebrafish, and the mammal may be a primate, a carnivore, an insectivore, a rodent, an artiodactyla, a perissodactyla, or an elephant, and the rodent may be a rat or a mouse.

According to the method, the fusion protein may further comprise a fluorescent protein. In this case, the fluorescent protein may fused to an N-terminus or to a C-terminus of the RTK or the light-induced dimerized protein fused to the RTK. The fluorescent protein is as described above.

According to the above-described embodiments, it is possible to regulate RTK signaling process in an animal cell or a plant cell specifically and reversibly. However, the scope of the present invention is not limited to the above-mentioned effects.

### BRIEF DESCRIPTION OF DRAWINGS

FIG. 1 is a schematic diagram illustrating the mechanism of a fusion protein activating RTK signaling by light:

RTK: receptor tyrosine kinase;

PHR: phytolyase homologous region which is an N-terminal region of chryptochrome protein; and

FP: fluorescent protein.

FIG. 2 represents a fluorescent microscopic image of cells taken with a confocal microscope showing the activation of MAPK signaling by light irradiation after expressing a fusion protein according to an embodiment of the present invention in the cells (a), and a graph showing the change in fluorescence intensity ratio of nucleus/cytosol over irradiation time (b).

FIG. 3 represents a fluorescent microscopic image of cells taken with a confocal microscope showing the activation of PI3K signaling by light irradiation after expressing a fusion protein according to an embodiment of the present invention in the cells (a), and a graph showing the change in fluorescence intensity in cytosol over irradiation time (b).

FIG. 4 is a fluorescent microscopic image of cells taken with a confocal microscope showing the reversible activation of calcium signaling by light irradiation after expressing a fusion protein according to an embodiment of the present invention in the cells.

FIG. 5 is a graph representing the result of the activation of calcium signaling of FIG. 4 as a change in fluorescence intensity over irradiation time.

FIG. 6 is a fluorescent microscopic image of cells taken with a confocal microscope showing that calcium signaling in cells was not inactivated by light irradiation when the cells were treated with a TrkB specific inhibitor after expressing a fusion protein according to an embodiment of the present invention in the cells.

FIG. 7 is a fluorescent microscopic image of cells taken with a confocal microscope showing that calcium signaling was activated only in specific cells expressing a fusion protein according to the present invention by light irradiation when co-culturing cells expressing the fusion protein according to an embodiment of the present invention (TrkB-PHR-YFP, R-GECO) and control cells not expressing the fusion protein (R-GECO).

### DETAILED DESCRIPTION OF THE INVENTION

Terms used in this document are defined as follows:

A “receptor tyrosine kinase” used in this document means a cell surface receptor binding to various peptide such as growth factors, cytokines and hormones.

A “light-induced dimerized protein” used in this document means a protein capable of forming a homodimer or a heterodimer with a partner protein when light irradiated.

A “partner protein” used in this document means a protein forming a heterodimer with the light-induced dimerized protein when light having particular wavelength is irradiated.

A “heterodimer” used in this document a complex formed by the interaction between different two proteins.

A “homodimer” used in this document means a complex formed by the interaction between two same proteins.

A “light-induced dimerized protein” used in this document means a protein capable of forming a homodimer or a heterodimer with a partner protein when light irradiated.

A “heterodimer” used in this document a complex formed by the interaction between different two proteins.

A “homodimer” used in this document means a complex formed by the interaction between two same proteins.

“Operably linked to” means that a particular polynucleotide or a particular polypeptide is linked to other polynucleotides or polypeptide, respectively in a manner that the particular polynucleotide or the particular polypeptide achieves its original function. In other words, “a polynucleotide encoding a particular polypeptide is operably linked to a promoter” means that the polynucleotide is linked to the promoter in order to be transcribed by the promoter and translated into the polypeptide, and “a polynucleotide encoding a particular polypeptide is operably linked to the other polynucleotide encoding a second polypeptide” means that the particular polypeptide is linked to the second polypeptide and so as to be expressed as a fusion protein retaining its original activity.

A “CIB” used in this document means a cryptochrome-interacting basic-helix-loop-helix protein and a representative CIB is Arabidopsis CIB1 (GenBank No. NM_{—}119618).

A “CIBN” used in this document means a N-terminal of the CIB, which is a part interacting with cryptochrome (CRY) when it is irradiated.

A “CRY” used in this document refers to a cryptochrome protein and a representative CRY is Arabidopsis CRY2 (GenBank No. NM_{—}100320).

A “PHR” used in this document refers to an N-terminal of the CRY which is a phytolyase homologous region of CRY and interacs with the CIB or the CIBN when it is irradiated (Kennedy et al., Nat. Methods, 7(12): 973-975, 2010).

A “Phy” used in this document refers to a phytochrome protein and a representative Phy is Arabidopsis PhyA (GenBank No.: NM_{—}001123784) and the Phy is known to interact with PIF (phytochrome interacting factor) (Min et al., Nature, 400: 781-784, 1999).

A “PIF” used in this document refers to a phytochrome interacting factor and a representative PIF includes Arabidopsis PIF1 (GenBank No.: NM_{—}001202630), PIF3 (GenBank No.: NM_{—}179295), PIF4 (GenBank No.: NM_{—}180050), PIF5 (GenBank No.: NM_{—}180690), PIF6 (GenBank No.: NM_{—}001203231) and PIF7 (GenBank No.: NM_{—}125520).

A “FKF” used in this document refers to a Flavin-binding, Kelch repeat, F-fox protein and a representative FKF is Arabidopsis FKF1 (GenBank No.: NM_{—}105475). It is known to interact with GIGANTEA protein when it is irraditated (Sawa et al., Science, 318 (5848): 261-265, 2007).

A “GIGANTEA” used in this document refers to a protein related to phytochrome signal transduction and is known to regulate flowering time of flowers.

A “tetracysteine motif” used in this document refers to a polypeptide containing an amino acid sequence of Cys-Cys-Xaa-Xaa-Cys-Cys (SEQ ID NO: 1), wherein the Xaa is any one amino acid except cysteine and fluofescent pattern varies depending on the type of Xaa and the length of the polypeptide (Adams et al., J. Am. Chem. Soc., 124: 6063-6077, 2002).

A “GECO” used in this document is an acronym representing “Genetically Encoded Calcium indicators for Optical imaging” and referred as a calcium sensing protein developed by Dr. Roger Campbell through random mutagenesis of GCaMP3, which is a fusion protein of calcium binding domain of calmodulin and a fluorescent protein. “R-GECO” is a calcium sensing protein based on a red fluorescent protein (RFP) and known that red fluorescence is increased about 16 folds when calcium binds to the protein (Zhao et al., Science, 333(6051): 1888-1891, 2011).

A “transgenic plant” or a “transgenic animal” used in this document are referred as a plant or an animal whose genomes are recombined to expressed a heterologous gene or to inactivate one or more intrinsic genes. Generally the transgenic animal may be prepared through a genetic manipulation of a germ cell, especially an oocyte or an embryonic stem cell. However, it may be prepared through a clonal replication by nuclear transfer method after genetic manipulation of a somatic cell. The transgenic plant can be more easily prepared. Specifically the transgenic plant may be prepared by infecting a plant cell with an agrobacterium containing a heterologous gene and then degenerating and regenerating the transformed plant cell. These methods for preparing the transgenic animals and plants are well known in the art (Jaenisch, R and B. Mintz, Proc. Natl. Acad. Sci. USA, 71 (4): 1250-1254, 1974; Cho et al., Curr. Protoc. Cell Biol., 42: 19.11.1-19.11.22, 2009; Johnston, S A and D C Tang, Meth. Cell Biol., 43 Pt A: 353-365, 1994; Sasaki et al., Nature 459 (7246): 523-527, 2009; Vaek et al., Nature, 328 (6125): 33-37, 1987).

Hereinafter, embodiments of the present inventions are described through the accompanying drawings. However, the present invention is not limited to the embodiments illustrated in the drawings and can be embodied as various embodiments, thus the embodiments illustrated in the drawings are provided in order to make the disclosure of the present invention complete and inform a skilled in the art the category of the present invention. In addition, components illustrated in the drawings can be exaggerated or downsized for convenience of description.

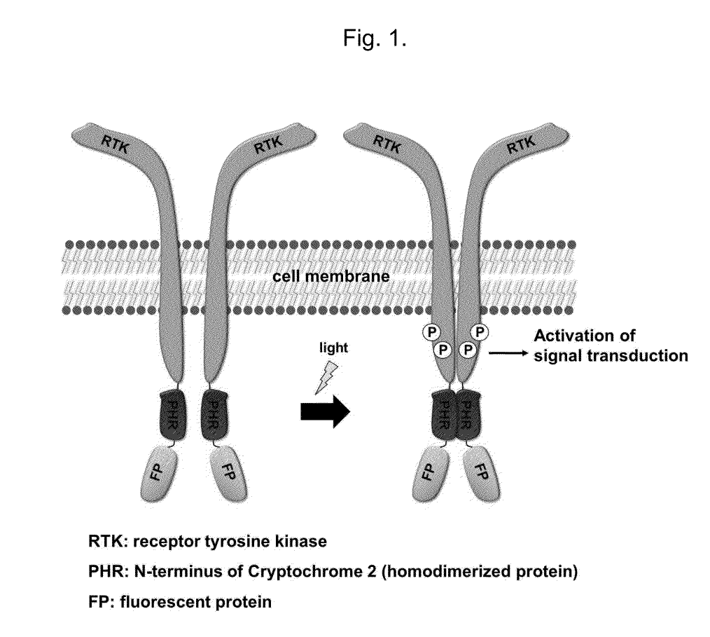

FIG. 1 is a schematic diagram illustrating the mechanism of a fusion protein activating RTK signaling by light. Generally RTKs dimerize, autophosphorylate and then activate various signaling pathways in a cell when ligands bind to extracellular domain of RTK. In the present invention, it is possible to regulate RTK signaling by light through regulating the dimerization of RTK depending on light irradiation using a fusion protein in which PHR, a plant photo receptor protein capable of forming a homodimer by light irradiation is fused to RTK.

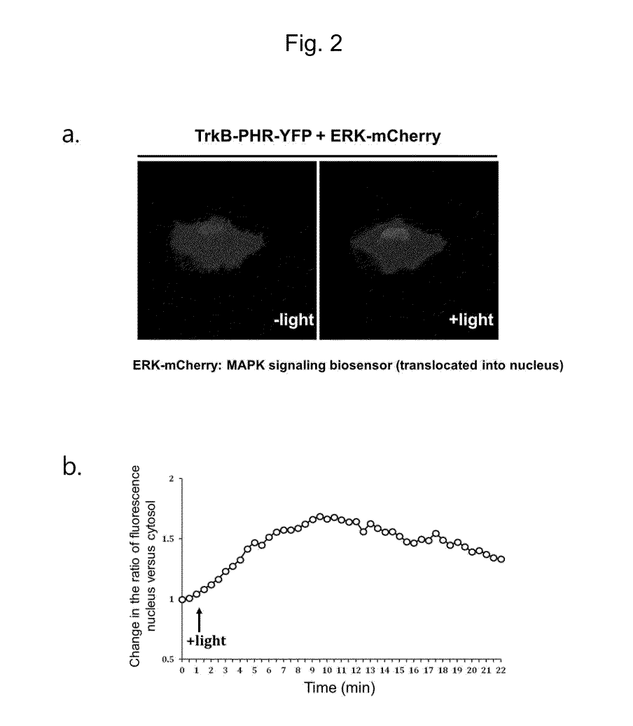

FIG. 2 represents a fluorescent microscopic image of cells taken with a confocal microscope showing the activation of MAPK signaling by light irradiation after expressing a fusion protein according to an embodiment of the present invention in the cells (a), and a graph showing the change in fluorescence intensity ratio of nucleus/cytosol over irradiation time (b). TrkB-PHR-YFP and ERK-mCherry constructs were prepared in order to investigate whether RTK is activated by light irradiation and MAPK signal pathway, downstream of RTK is activated normally. Fluorescence intensity of nucleus/cytosol got increased over time when light is irradiated after transfecting a cell with the constructs. This phenomenon was resulted from the translocation of ERK present in the cytosol into the nucleus when MAPK signaling was activated and proved that TrkB-MAPK signaling was activated by light irradiation.

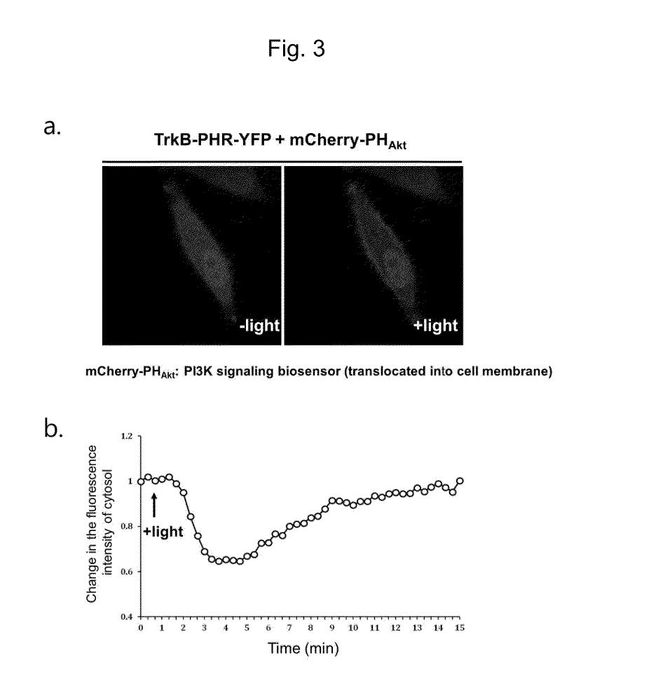

FIG. 3 represents a fluorescent microscopic image of cells taken with a confocal microscope showing the activation of PI3K signaling by light irradiation after expressing a fusion protein according to an embodiment of the present invention in the cells (a), and a graph showing the change in fluorescence intensity in cytosol over irradiation time (b). TrkB-PHR-YFP and mCherry-PHₐₖₜ constructs were prepared in order to investigate whether RTK and PI3K signal pathways, downstream of the RTK are activated by light irradiation normally. It was observed that fluorescence intensity of cytosol got decreased but the signal of fluorescence moved to cell membrane over time when light is irradiated after transfecting a cell with the constructs. This phenomenon was resulted from the translocation of PH domain of Akt present in the cytosol into the cell membrane and the interaction between the PH domain and PIP3. Thus it was proved that TrkB-PI3K signaling was activated by light irradiation.

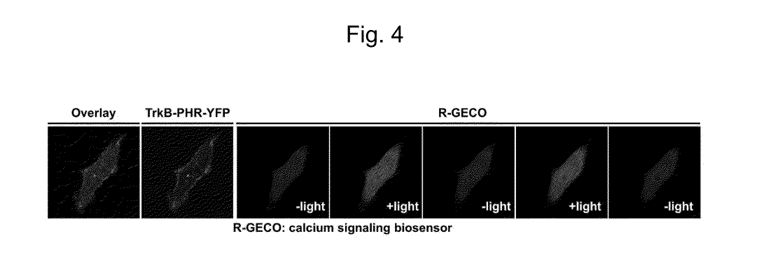

FIG. 4 is a fluorescent microscopic image of cells taken with a confocal microscope showing the reversible activation of calcium signaling by light irradiation after expressing a fusion protein according to an embodiment of the present invention in the cells. TrkB-PHR-YFP was prepared as described above and R-GECO construct was purchased in order to investigate whether RTK and calcium signaling pathway, downstream of the RTK are activated by light irradiation normally. As a result, it was observed that fluorescence intensity of cytosol got increased over time when light is irradiated after transfecting a cell with the constructs which means that influx of calcium to the cytosol increased. On the other hand, the fluorescence intensity decreased when the light irradiation was stopped. Thus it was proved that it is possible to regulate calcium signaling reversibly by light irradiation.

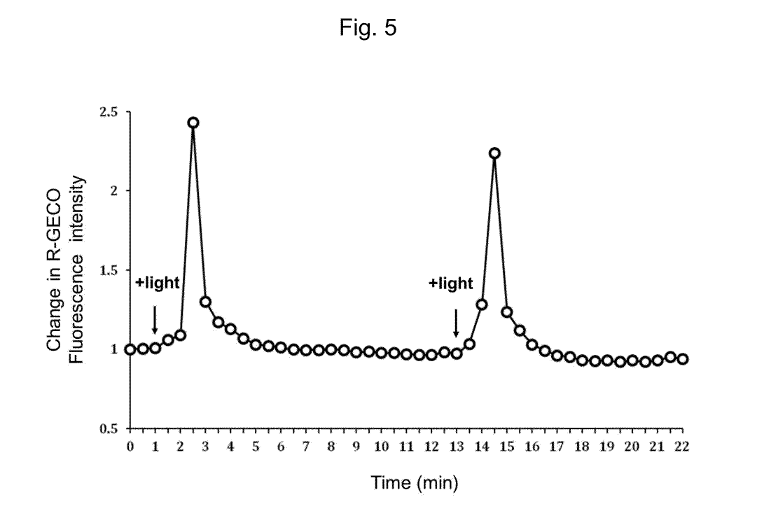

FIG. 5 is a graph representing the result of the activation of calcium signaling of FIG. 4 as a change in fluorescence intensity over irradiation time. The graph is illustrated with an arbitrary unit of calcium influx level defining the level of fluorescence intensity when no light is irradiated as reference (“1”).

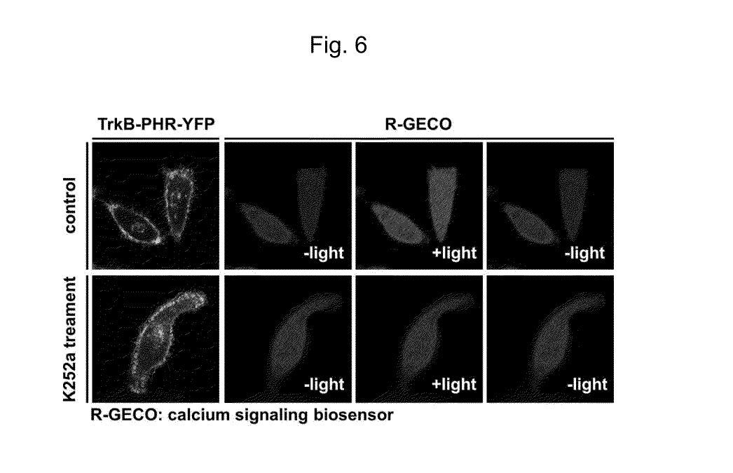

FIG. 6 is a fluorescent microscopic image of cells taken with a confocal microscope showing that calcium signaling in cells was not inactivated by light irradiation when the cells were treated with a TrkB specific inhibitor after expressing a fusion protein according to an embodiment of the present invention in the cells. Cells transfected with the TrkB-PHR-YFP and the R-GECO constructs were treated with a TrkB specific inhibitor, K252a and then light irradiated. As a result, it was observed that there is no difference in the experimental group treated with K252a. On the contrary, the calcium signaling was changed according to light irradiation in the control group. This result suggests that the fusion protein according to an embodiment of the present invention activates only RTK specific signaling.

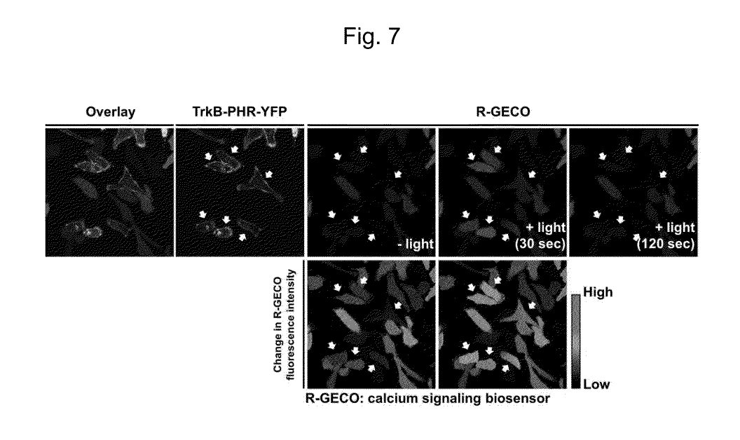

FIG. 7 is a fluorescent microscopic image of cells taken with a confocal microscope showing that calcium signaling was activated only in specific cells expressing a fusion protein according to the present invention by light irradiation when co-culturing cells expressing the fusion protein according to an embodiment of the present invention (TrkB-PHR-YFP, R-GECO) and control cells not expressing the fusion protein (R-GECO). As shown in FIG. 7, it was observed that calcium signaling was activated only in cells expressing TrkB-PHR-YFP.

Therefore, the fusion protein according to an embodiment of the present invention may be applied to a tissue level model or an animal model and be used for investigating the mechanism related to RTK signaling, and the development, growth and differentiation of cells, and behavioral analysis of animal model, etc.

Hereinafter, the present invention is described in detail through accompanying examples and experimental examples. However, the present invention is not limited to the examples and the experimental examples and may be embodied as various embodiments, thus the examples and the experimental examples are provided in order to make a more complete disclosure of the present invention and inform a skilled in the art the category of the present invention.

### EXAMPLE 1

### Preparation of Vectors

### 1-1: Construction of TrkB-PHR-YFP Construct

The TrkB-PHR-YFP was constructed by preparing a polynucleotide encoding a fusion protein in which PHR domain (1-498 a.a.) of CRY2 (GenBank No.: NM_{—}100320) is fused to the C-terminus of TrkB (GenBank No.: NM_{—}00116368) and inserting the polynucleotide to the N-terminus of mCitrine-N1 vector prepared based on EGFP-N1 vector.

### 1-2: Construction of ERK-mCherry Construct

The ERK-mCherry construct was constructed by inserting a polynucleotide encoding a full length ERK (GenBank No.: NM_{—}011952) to the multicloning site of pmCherry-N1 vector (Clontech, USA) in frame.

### 1-3: Construction of mCherry-PH_{Akt} Construct

The mCherry-pPH_{Akt} construct was constructed by inserting a polynucleotide encoding a PH domain (2-147 a.a.) of Akt (GenBank No.: NM_{—}001014431) to the multicloning site of pmCheery-C1 vector (Clontech, USA).

### EXPERIMENTAL EXAMPLE 1

### Activation of Downstream Signaling Pathway of TrkB by Light Irradiation

The present inventors observed whether MAPK, PI3k and calcium signaling is activated in order to confirm that the fusion protein according to an embodiment of the present invention activates downstream signaling of TrkB by light irradiation.

First, HeLa cells were co-transfected with the TrkB-PHR-YFP construct and the ERK-mCherry construct prepared in the examples 1-1 and 1-2, respectively and then cultured in DMEM media containing 10% FBS in the condition of 37° C. and 10% CO₂. The co-transfected HeLa cells were imaged with a confocal microscope before and after the irradiation with light having 488 nm of wavelength (FIG. 2a) and then the ratio of the change of fluorescence intensity within nucleus/cytosol was calculated according to the measurement of the change of fluorescence intensity in the nucleus and the cytosol over time (FIG. 2b). As a result, it was observed that the fluorescence in the cytosol got stronger due to the light irradiation, and this phenomenon was resulted from the translocation of activated ERKs into. This result suggests that MAPK signaling which is a downstream signaling of the TrkB was activated by the light irradiation.

Subsequently, the present inventors investigated whether PI3K, a representative downstream signaling molecule of the TrkB was activated by the light irradiation in order to determine whether the fusion protein according to an embodiment of the present invention can activate downstream signaling of the TrkB. For this, HeLa cells were co-transfected with the TrkB-PHR-YFT construct and the mCherry-PH_{Akt} construct prepared in the examples 1-1 and 1-3, respectively and then cultured in DMEM media containing 10% FBS in the condition of 37° C. and 10% CO₂. The co-transfected HeLa cells were imaged with a confocal microscope before and after the irradiation with light having 488 nm of wavelength (FIG. 3a) and then the change of fluorescence intensity in the cytosol over time (FIG. 3b). As a result, it was observed that the fluorescence of mCherry-PH_{Akt} construct moved gradually into the cell membrane. This phenomenon is observed when the PH domain of Akt is translocated into the cell membrane and interacted with PIP3 when PI3K signaling is activated and this result suggests that TrkB-PI3K signaling process was activated by the light irradiation.

Further, the present inventors investigated whether the fusion protein according to an embodiment of the present inventior can activate calcium signaling, a representative downstream signaling of the TrkB. For this, HeLa cells were co-transfected with TrkB-PHR-YFP construct prepared in the example 1-1 and R-GECO construct (Addgene, plasmid 32444; CMV-R-GECO1) and then cultured in DMEM media containing 10% FBS in the condition of 37° C. and 10% CO2. The co-transfected HeLa cells were imaged with a confocal microscope before and after the irradiation with light having 488 nm of wavelength (FIG. 4) and then the fluorescence intensity of R-GECO in the cytosol (FIG. 5). The R-GECO is a biosensor capable of monitoring calcium signaling which is a downstream signaling pathway of TrkB, and was used for measuring the increase or decrease of intracellular calcium level. As a result, it was observed that the fluorescence of R-GECO got increased within the cytosol but, it was decreased when the light irradiation was removed. Also, as shown in FIG. 5, when the light was irradiated repeatedly, the fluorescence of R-GECO got stronger in a synchronous pattern. This result suggests that TrkB activity can be induced by the light irradiation.

Therefore, the present inventors revealed that the fusion protein according to an embodiment of the present invention is activated by light irradiation and downstream signaling of TrkB which is a member of RTKs is activated thereby. This result is a selective result using an examplery TrkB among RTKs, and thus the present invention may be used for studies on the mechanism of RTK-related signal trusduction, the development, growth and differentiation of cells, and behavioral analysis of animal model, etc. using various other RTKs besides the TrkB.

### EXPERIMENTAL EXAMPLE 2

### Activation of TrkB Specific Signal Transduction Induced by Light Irradiation

The present inventors observed whether sub-RTK signaling was activated by light irradiation after treating a RTK-specific inhibitor in order to confirm that only RTK-specific signaling pathways are activated by light irradiation affecting downstream pathways thereof.

The present inventors used TrkB-PHR-YFP construct prepared in the example 1-1 as an example to investigate the activation of TRK signal transduction. In addition, the present inventors used R-GECO construct (Addgene, plasmid 32444; CMV-R-GECO1) to investigate whether calcium signaling among downstream signaling pathways of RTK is activated by light irradiation. Particularly, HeLa cells were co-transfected with the TrkB-PHR-YFP construct prepared in the example 1-1 and R-GECO construct and cultured in DMEM media containing 10% FBS in the condition of 37° C. and 10% CO₂. The cultivated cells were treated with 100 nM of K252a known as a TrkB-specific inhibitor and images taken with a confocal microscope before and after the irradiation with light having 488 nm of wavelength were obtained and analyzed (FIG. 6). As a result, it was observed that there is no difference in the experimental group treated with K252a. On the contrary, the calcium signaling was changed according to light irradiation in the control group. This result suggests that the fusion protein according to an embodiment of the present invention activates only RTK specific signaling.

In addition, the present inventors co-cultured cells co-expressing the construct of the example 1-1 and R-GECO construct (Addgene, plasmid 32444; CMV-R-GECO1) and cells expressing only R-GECO construct at a ratio of 2:1 in order to investigate whether the fusion protein according to an embodiment of the present invention can be applied in a tissue level. Confocal microscopic images were obtained before and after irradiating light having 488 nm of wavelength from cells co-transfected with the construct of example 1-1 and the R-GECO construct (1:1 ratio, each 300 nm) (FIG. 7). As a result, the fluorescence of R-GECO only in cells expressing the TrkB-PHR-YFP construct and the R-GCO construct (arrow of FIG. 7). This suggests that the system according to the present invention can be applied to the activation of RTK signaling by light irradiation at a level of tissue containing various types of cells.

Therefore, the fusion protein according to an embodiment of the present invention may be used for studies on the mechanism related to RTK signaling, and the development, growth and differentiation of cells, and behavioral analysis of animal model, etc.

While the present invention has been described in connection with certain exemplary examples, it is to be understood that the invention is not limited to the disclosed examples, but, on the contrary, is intended to cover various modifications and equivalent arrangements included within the spirit and scope of the appended claims, and equivalents thereof.

### SEQUENCE LISTING FREE TEXT

SEQ ID No. 1 is an amino acid sequence of tetracysteine motif.

1. A fusion protein in which a light-induced dimerized protein is fused to C-terminus of receptor tyrosine kinase (RTK) or RTK mutant protein whose ligand binding domain is deleted or mutated so as not to bind to a ligand.

2. The fusion protein according to claim 1, wherein the receptor tyrosine kinase is epidermal growth factor receptor (EGFR, RTK class I), insulin receptor (IR, RTK class II), platelet-derived growth factor receptor (PDGF, RTK class Ill), fibroblast growth factor receptor (FGFR, RTK class IV), vascular endothelial cell growth factor receptor (VEGFR, RTK class V), hepatocyte growth factor receptor (HGFR, RTK class VI), tropomyosin-receptor-kinase receptor (TRKR, RTK class VII), EPH receptor (RTK class VIII), AXL receptor (RTK class IX), LKT receptor (RTK class X), TIER receptor (RTK class XI), receptor tyrosine kinase-like orphan receptor (RTK class XII), discoidin domain receptor (RTK class XIII), RET receptor (RTK class XIV), KLG receptor (RTK class XV), related to receptor tyrosine kinase (RYK) receptor (RTK class XVI), or Muscle-Specific Kinase (MuSK) Receptor (RTK class XVII).

3. The fusion protein according to claim 1, wherein the light-induced dimerized protein is a light-induced heterodimerized protein or a light-induced homodimerized protein.

4. The fusion protein according to claim 2, wherein the light-induced heterodimerized protein is CIB (cryptochrome-interacting basic-helix-loop-helix protein), CIBN (N-terminal domain of CIB), Phy (phytochrome), PIF (phytochrome interacting factor), FKF1 (Flavin-binding, Kelch repeat, F-box 1 GIGANTEA, CRY (chryptochrome) or PHR (phytolyase homolgous region).

5. The fusion protein according to claim 3, wherein light-induced homodimerized protein is CRY or PHR.

6. The fusion protein according to claim 3, further comprising a fluorescent protein.

7. The fusion protein according to claim 6, wherein the fluorescent protein is green fluorescent protein (GFP), yellow fluorescent protein (YFP), red fluorescent protein (RFP), orange fluorescent protein, cyan fluorescent protein (CFP), blue fluorescent protein (BFP), or tetracysteine fluorescent motif.

8. A polynucleotide encoding the fusion protein of claim 1.

9. A vector comprising the polynucleotide of claim 8.

10. A transformed host cell prepared by the transformation of a host cell with the vector of claim 9.

11. A non-human transgenic animal transformed with the vector of claim 9 and capable of expressing the fusion protein.

12. A transgenic plant transformed with the vector of claim 9 and capable of expressing the fusion protein.

13. A method for activating RTK in a cell reversibly, comprising:
preparing a transformed host cell by transforming a host cell with an expression vector comprising a gene construct including a promoter and a polynucleotide encoding the fusion protein of claim 1, wherein the polynucleotide is operably linked to the promoter; and
irradiating light having wavelength capable of inducing the homodimerization of the light-induced homodimerized protein.

14. A method for activating receptor tyrosine kinase (RTK) in an organ or a tissue of a plant or a non-human animal, comprising:
preparing a transgenic plant or a transgenic non-human animal by transforming a plant or a non-human animal with an expression vector comprising a gene construct including a promoter and a polynucleotide encoding the fusion protein of claim 1, wherein the polynucleotide is operably linked to the promoter; and
irradiating light having wavelength capable of inducing the homodimerization of the light-induced homodimerized protein to the organ or the tissue.

15. The method according to claim 13, wherein the RTK is epidermal growth factor receptor (EGFR, RTK class I), insulin receptor (IR. RTK class II), platelet-derived growth factor receptor (PDGF, RTK class III), fibroblast growth factor receptor (FGFR, RTK class IV), vascular endothelial cell growth factor receptor (VEGFR, RTK class V), hepatocyte growth factor receptor (HGFR, RTK class VI), tropomyosin-receptor-kinase receptor (TRKR, RTK class VII), EPH receptor (RTK class VIII), AXL receptor (RTK class IX), LKT receptor (RTK class X), TIER receptor (RTK class XI), receptor tyrosine kinase-like orphan receptor (RTK class XII), discoidin domain receptor (RTK class XIII), RET receptor (RTK class XIV), KLG receptor (RTK class XV), related to receptor tyrosine kinase (RYK) receptor (RTK class XVI), or Muscle-Specific Kinase (MuSK) Receptor, RTK class XVII).

16. The method according claim 13, wherein the light-induced homodimerized protein is cryptochrome (CYR) or PHR (phytolyase homologous region).

17. The method according to claim 13, wherein the host cell is an animal cell or a plant cell.

18. The method according to claim 13, wherein the fusion protein further comprises a fluorescent protein.

19. The method according to claim 18, wherein the fluorescent protein is green fluorescent protein (GFP), yellow fluorescent protein (YFP), red fluorescent protein (RFP), orange fluorescent protein, cyan fluorescent protein (CFP), blue fluorescent protein (BFP), or tetracysteine fluorescent motif.

20. A method for screening an inhibitor candidate of RTK signaling comprising:
preparing a transformed host cell by transforming a host cell with an expression vector comprising a gene construct including a promoter and a polynucleotide encoding the fusion protein of claim 1, wherein the polynucleotide is operably linked to the promoter;
treating candidate substances to culture media containing the transformed cell;
irradiating light having wavelength capable of inducing the homodirnerization of the light-induced homodimerized protein to the transformed cell; and
selecting a candidate substance inhibiting RTK signaling by the light irradiation significantly compared to a negative control,

21. The method according to claim 20, wherein the RTK is epidermal growth factor receptor (EGFR, RTK class I), insulin receptor (IR, RTK class II), platelet-derived growth factor receptor (PDGF, RTK class III), fibroblast growth factor receptor (FGFR, RTK class IV), vascular endothelial cell growth factor receptor (VEGFR, RTK class V), hepatocyte growth factor receptor (HGFR, RTK class VI), tropomyosin-receptor-kinase receptor (TRKR, RTK class VII), EPN receptor (RTK class VIII), AXL receptor (RTK class IX), LKT receptor (RTK class X), TIER receptor (RTK class XI), receptor tyrosine kinase-like orphan receptor (RTK class XII), discoidin domain receptor (RTK class XIII), RET receptor (RTK class XIV), KLG receptor (RTK class XV), related to receptor tyrosine kinase (RYK) receptor (RTK class XVI), or Muscle-Specific Kinase (MuSK) Receptor, RTK class XVII).

22. The method according to claim 20, wherein the candidate substance is a peptide, a protein, a non-peptide compound, a synthetic compound, a fermentation product, cell extract, plant extract, animal tissue extracts or plasma.

23. The method according to claim 20, wherein the light-induced homodimerized protein is CRY or PHR.

24. The method according to claim 20, wherein the host cell is an animal cell or a plant cell.

25. The method according to claim 20, wherein the fusion protein further comprises a fluorescent protein.

26. The method according to claim 25, wherein the fluorescent protein is green fluorescent protein (GFP), yellow fluorescent protein (YFP), red fluorescent protein (RFP), orange fluorescent protein, cyan fluorescent protein (CFP), blue fluorescent protein (BFP), or tetracysteine fluorescent motif.

27. The method according to claim 14, wherein the RTK is epidermal growth factor receptor (EGFR, RTK class I), insulin receptor (IR, RTK class II), platelet-derived growth factor receptor (PDGF, RTK class III), fibroblast growth factor receptor (FGFR, RTK class IV), vascular endothelial cell growth factor receptor (VEGFR, RTK class V), hepatocyte growth factor receptor (HGFR, RTK class VI), tropomyosin-receptor-kinase receptor (TRKR, RTK class VII), EPH receptor (RTK class VIII), AXL receptor (RTK class IX), LKT receptor (RTK class X), TIER receptor (RTK class XI), receptor tyrosine kinase-like orphan receptor (RTK class XII), discoidin domain receptor (RTK class XIII), RET receptor (RTK class XIV), KLG receptor (RTK class XV), related to receptor tyrosine kinase (RYK) receptor (RTK class XVI), or Muscle-Specific Kinase (MuSK) Receptor, RTK class XVII).

28. The method according claims 14, wherein the light-induced homodimerized protein is cryptochrome (CYR) or PHR (phytolyase homologous region).

29. The method according to claim 14, wherein the fusion protein further comprises a fluorescent protein.

30. The method according to claim 29, wherein the fluorescent protein is green fluorescent protein (GFP), yellow fluorescent protein (YFP), red fluorescent protein (RFP), orange fluorescent protein, cyan fluorescent protein (CFP), blue fluorescent protein (BFP), or tetracysteine fluorescent motif.


SEQUENCE LISTING (1 sequence)

SEQ ID NO: 1 - protein, 6 aa, Artificial Sequence
  tetracystein motif
CCXXCC